(19) United States
(12) Patent Application Publication
(10) Pub. No.: US 2022/0370274 A1
(43) Pub. Date: Nov. 24, 2022

(54) FIELD-TYPE MODULAR NEGATIVE PRESSURE ISOLATION ROOM SYSTEM AND METHOD FOR CONSTRUCTING FIELD-TYPE MODULAR NEGATIVE PRESSURE ISOLATION ROOM SYSTEM

(71) Applicant: Jong Jin Park, Gyeonggi-do (KR)

(72) Inventor: Jong Jin Park, Gyeonggi-do (KR)

(21) Appl. No.: 17/630,357

(22) PCT Filed: Dec. 21, 2020
(86) PCT No.: PCT/KR2020/018775

§ 371 Date: Jan. 26, 2022

(30) Foreign Application Priority Data
Oct. 15, 2020 (KR) 10-2020-0133249

(51) Int. Cl. A61G 10/02; A61G 10/00; E04B 1/343; E04B 1/61; E04B 1/41; E04H 1/12; E04H 3/08

(52) U.S. Cl. CPC A61G 10/023; A61G 10/005; E04B 1/34321; E04B 1/6108; E04B 1/40; E04H 1/1277; E04H 3/08; E04H 1/1205; E04B 2001/405

(57) ABSTRACT

The present disclosure relates to a field-type negative pressure isolation room system including main facilities necessary for constructing a negative pressure isolation room defined by the legal regulations, wherein the main facilities are manufactured in a plant in a modular way, so these modular facilities are usually stored in a warehouse, etc., and are installed in an outdoor site such as a parking lot, playground, vacant lot, and the like in case of emergency to utilize as a negative pressure isolation room, and these modular facilities are disinfected and dismantled during demolition and stored in the warehouse again, whereby these modular facilities can be used repeatedly regardless of the number of times of use, and can be quickly constructed and dismantled.

### TECHNICAL FIELD

The present disclosure relates to a field-type negative pressure isolation room system, and more particularly, to a field-type negative pressure isolation room system including main facilities necessary for constructing a negative pressure isolation room defined by the legal regulations, wherein the main facilities are manufactured in a plant in a modular structure, so these modular facilities are usually stored in a warehouse, etc., and are installed in an outdoor site such as a parking lot, playground, vacant lot, and the like in case of emergency to utilize as a negative pressure isolation room, and these modular facilities are disinfected and dismantled during demolition and stored in the warehouse again, whereby these modular facilities can be used repeatedly regardless of the number of times of use, and can be quickly constructed and dismantled.

### BACKGROUND ART

In general, respiratory infectious diseases such as Midwestern respiratory syndrome (MERS), severe acute respiratory syndrome (SARS), anthrax, Ebola hemorrhagic fever, and the like are diseases that require immediate isolation measures, because it is not easy to predict the infection rout thereof and the rate of spread thereof is very fast.

SARS, which was originated in Southeast Asia in 2003, had been spread to Europe, North America, and Asia, and about 83,000 peoples were infected with SARS, 10% of them died. Then, the seriousness of infectious diseases has been emerged around the world through the novel swine-origin influenza in 2009 and the MERS in 2015, and it is urgent to expand the isolation rooms since many of the confirmed cases were infected in hospitals.

However, current negative pressure isolation rooms have been placed in some facilities of large hospitals, and considering regional location and volume of large hospitals, it is predicted that when a confirmed case occurs within a large hospital, the rate of infection spread will be unpredictably fast.

As of 2020, the number of negative pressure isolation rooms in Republic of Korea is about 2,000, including single room and multi-person rooms, and corona virus disease 19 (COVID-19), which occurred in 2019, has been spreading all over the world (a global pandemic), and the demand for negative pressure isolation sickbeds and negative pressure isolation rooms is absolutely insufficient, thus raising the problem of facility expansion.

Although there is an absolute shortage of negative pressure isolation rooms for patient treatment and isolation as above, when newly constructing negative pressure isolation rooms, more than six months of construction time and huge maintenance costs occur, so there is a problem in that it is difficult to maintain realistically negative pressure isolation rooms in existing hospitals.

The negative pressure isolation room is a medical structure constructed such that, when accommodating and treating infected patients separately from outsiders and general patients for reasons such as preventing the spread of infectious diseases, air pressure in the room is kept lower than the atmospheric pressure to prevent pathogens contained in air, aerosol or droplets inside the room from being discharged to the outside, and air in the negative pressure isolation room is exhausted to the outside through an exhaust facility (to which a highly efficient particulate air filter is applied to prevent pathogens from passing through).

The negative pressure isolation room should be provided with a front chamber which is a space where medical staff wearing protective suits can disinfect, and is configured such that the absolute value of negative pressure is maintained in the order of toilet→room→front chamber→hallway to prevent air in the toilet from entering the room or the front chamber even when a door is opened, a direction of air flow is designed to prevent air in the room from entering the front chamber, and a negative pressure state may be maintained.

In a basic structure of the typical negative pressure isolation room, a hallway, a front chamber, a room and a toilet are arranged in order such that an absolute value of negative pressure is increased in that order, in general, an exhaust unit is located in an area adjacent to a bedside of a bed, which is located far from a door of the room and an air supply unit is located in an area adjacent to the door, and a pressure gauge is installed in the front chamber to check the air pressure.

In addition, although a FFU (fan filter unit) through which an exhaust unit is communicated may be located inside or outside a room, this FFU may be preferably disposed outside the room from the standpoint of maintenance, and is typically configured to be sterilized and disinfected as well as sterilized by dust collection and adsorption.

The negative pressure isolation rooms are employed to prevent the spread of emerging infectious diseases COVID 19, Midwestern respiratory syndrome (MERS), severe acute respiratory syndrome (SARS), avian influenza human infection (AIHI), novel swine-origin influenza A (H1N1), Ebola hemorrhagic fever, and the like.

On the other hand, the bacterial contamination preventing system for the bio-clean room disclosed in Korea Utility Model Registration No. 20-0406536, registered on Jan. 12, 2006, includes a differential pressure device having an air differential pressure sensor formed therein and controlling an air differential pressure in an isolation room, and a control unit for controlling the differential pressure device, and this system utilizes an air differential pressure device employed in the isolation room in which an infectious case is isolated, an inlet pipe into which air flows and an outlet pipe are formed on the isolation chamber.

In a conventional negative pressure isolation room, however, since enormous cost and time for its installation are required, there is a serious problem in that it is difficult to quickly counteract to the spread of emerging infectious disease, and if there is a problem in the management of the medical staff, there is a risk of further spreading infection within the same ward, furthermore, although the principle of using one room per person should be applied for patient with emerging infectious disease, there is a problem in that a room is often operated for 2 to 6 peoples due to cost concerns.

### DISCLOSURE OF THE INVENTION

### Technical Problem

The present disclosure is invented for improving the above-described problems, and the first object to be solved by the present disclosure is to provide a field-type modular negative pressure isolation room system including main facilities necessary for constructing a negative pressure isolation room defined by the legal regulations, wherein the main facilities are manufactured in a plant in a modular way, so these modular facilities are usually stored in a warehouse, etc., and are installed in an outdoor site such as a parking lot, playground, vacant lot, and the like in case of emergency to utilize as a negative pressure isolation room, and these modular facilities are disinfected and dismantled during demolition and stored in the warehouse again, whereby these modular facilities can be used repeatedly regardless of the number of times of use, and can be quickly constructed and dismantled, and a method for constructing a field-type modular negative pressure isolation room system.

Also, the second object of the present disclosure is to provide a field-type modular negative pressure isolation room system which enables rapid and efficient isolation, treatment and observation for confirmed cases or suspected infections by early constructing a negative pressure room for patients with emerging infectious diseases outside a ward, and can be constructed at a relatively low to have highly cost, thereby having excellent economic feasibility, and a method for constructing the field-type modular negative pressure isolation room system.

In addition, the third object of the present disclosure is to provide a field-type modular negative pressure isolation room system in which there is no risk of further spread of infection due to indoor transmission in the same ward, and a method for constructing the field-type modular negative pressure isolation room system.

Furthermore, the fourth object of the present disclosure is to provide a field-type modular negative pressure isolation room system having excellent maintainability because, although having a portable type, it is easy to access a machine room in which a HEPA filter unit (HFU) is accommodated, and a method for constructing the field-type modular negative pressure isolation room system.

Also, the fifth object of the present disclosure is to provide a field-type modular negative pressure isolation room system which may transport an infected person using a trailer in a state in which the infected person is isolated, thereby fundamentally blocking the risk of further infection due to the transport of the infected patient, and a method for constructing the field-type modular negative pressure isolation room system.

In addition, the sixth object of the present disclosure is to provide a field-type modular negative pressure isolation room system which has a drainage facility isolated from the outside, although having a movable structure, and seeks energy-saving energy independence, and a method for constructing the field-type modular negative pressure isolation room system.

Furthermore, the seventh object of the present disclosure is to provide a field-type modular negative pressure isolation room system which may easily apply the principle of single occupancy for a confirmed case or suspected infection of emerging infectious disease, and a method for constructing the field-type modular negative pressure isolation room system.

The technical object of the present disclosure is not limited to those mentioned above, and another technical problem not mentioned will be clearly understood by those skilled in the art from the following description.

### Technical Solution

In order to achieve the above object, a field-type modular negative pressure isolation room system according to the first embodiment of the present disclosure is characterized in that an inner wall panel and an outer wall panel are installed between a floor panel and a ceiling panel to partition an internal space into a plurality of rooms, and the floor panel, the ceiling panel, the inner wall panel and the outer wall panel are assembled in a modular structure which may be dismantled.

Also, the plurality of rooms may include a front chamber, a negative pressure isolation room, a toilet, and a mechanical equipment room, or may include a vestibule for a hallway, a shower room and toilet for a medical staff, a sewage/waste disposal room, a utility and equipment storage room, a negative pressure hallway, an online visiting room, a negative pressure isolation room, a front chamber for a room, a toilet for a patient, a mechanical equipment room.

In addition, the floor panel includes a plurality of base plates assembled adjacent to each other in a horizontal direction; a plurality of floor plates installed on the base plate and assembled adjacent to each other in the horizontal direction; and a lower end fixing plate installed between the base plate and the floor plate, and having a fixing groove part formed at an edge portion by bending the lower end fixing plate so as to allow lower end portions of the inner wall panel and the outer wall panel to be inserted and coupled thereto.

Furthermore, the ceiling panel includes a plurality of roof plates positioned on an uppermost surface and assembled adjacent to each other in the horizontal direction; a plurality of ceiling plates installed under the roof plate and assembled adjacent to each other in the horizontal direction; and an upper end fixing plate installed between the roof plate and the ceiling plate, and having a fixing groove part formed at an edge portion by bending the upper end fixing plate so as to allow upper end portions of the inner wall panel and the outer wall panel to be inserted and coupled thereto.

Also, a plurality of reinforcement wall panels may be installed on an outer surface of the outer wall panel by a wall panel reinforcing-connecting means.

In addition, the wall panel reinforcing-connecting means includes a connection block that is fixedly installed on any one of both outer surfaces of the reinforcement wall panel; a “ [symbol] ”-shaped connection bracket fixedly installed on the other one of both outer surfaces of the reinforcement wall panel so as to be coupled to the connection block; and a fastening bolt passing through the “ [symbol] ”-shaped connection bracket and the connection block to fasten them to each other in a state in which the connection block is inserted in a coupling groove of the “ [symbol] ”-shaped connection bracket.

Furthermore, air supply/exhaust facilities may be alternately arranged in a zigzag form on the ceiling panel.

A seal member may be installed at a connecting portion between the inner wall panels, a connecting portion between the outer wall panels, and a connecting portion between the inner wall panel and the outer wall panel.

Meanwhile, the field-type modular negative pressure isolation room system according to the second embodiment of the present disclosure further includes a bracket for connecting the ceiling panel and the wall panel installed under the ceiling panel so as to assemble the inner wall panel and the outer wall panel to the ceiling panel.

The bracket for connecting the ceiling panel and the wall panel includes a first channel part configured to allow an upper end portion of the outer wall panel to be inserted thereto; a coupling protrusion extending on an upper surface of the first channel part to be coupled into a coupling hole of the ceiling panel; and a second channel part formed perpendicular to the first channel part to allow an upper end portion of the inner wall panel to be inserted thereinto.

The bracket for connecting the ceiling panel and the wall panel has a T shape.

Meanwhile, a method for constructing the field-type modular negative pressure isolation room system according to the first embodiment of the present disclosure includes the steps of performing planarization for a bottom surface; installing a base truss on the bottom surface; installing the floor panel on the base truss; coupling the lower end portions of the inner wall panel and the outer wall panel into the fixing groove part formed in the lower end fixing plate of the floor panel and installing vertically the inner wall panel and the outer wall panel to partition an internal space into the plurality of rooms; and installing the ceiling panel to which the air supply/exhaust facility and the HEPA filter are mounted, on the upper end portions of the inner wall panel and the outer wall panel, and inserting and coupling the upper end portions of the inner wall panel and the outer wall panel into the fixing groove part formed in the upper end fixing plate of the ceiling panel.

In addition, a method for constructing the field-type modular negative pressure isolation room system according to the second embodiment of the present disclosure includes the steps of performing planarization for the bottom surface; installing the base truss on the bottom surface; installing the floor panel on the base truss; coupling the lower end portions of the inner wall panel and the outer wall panel into the fixing groove part formed in the lower end fixing plate of the floor panel and installing vertically the inner wall panel and the outer wall panel to partition an internal space into the plurality of rooms; and installing the ceiling panel to which the air supply/exhaust facility and the HEPA filter are mounted, on the upper end portions of the inner wall panel and the outer wall panel, and coupling the upper end portions of the inner wall panel and the outer wall panel to the ceiling panel using the bracket for connecting the ceiling panel and the wall panel.

In the present disclosure, the inner space is partitioned into the plurality of rooms by assembling the floor panel, the ceiling panel, and the inner wall panel, and the outer wall panel, wherein the floor panel, the ceiling panel, the inner wall panel, and the outer wall panel are configured as the module, so assembly and disassembly (dismantlement) for the above panels may be repeatedly performed.

### Advantageous Effects

As described above, the present disclosure has effects as below.

Firstly, the above-mentioned negative pressure isolation room is a negative pressure isolation room which can be quickly installed for preparing the spread of infectious diseases and disassembled, as compared to a conventional negative pressure isolation room installed in a hospital, a construction cost therefor is low, and concerns about secondary infection in a hospital is low.

Secondly, the above-mentioned negative pressure isolation room is a “mobile modular negative pressure room” which can be installed once a certain space is secured, and can be installed and dismantled in a region with low population density, so it is possible to minimize the occurrence of nimby phenomena, such as resistance of the residents caused by designating the existing facility as a “designated facility (hospital) for COVID 19”.

Thirdly, all main facilities necessary for constructing the negative pressure isolation room defined by the legal regulations are manufactured in a plant in a modular way, so the production process for the facilities is fast, and these facilities are normally dismantled and stored in warehouses, etc., and can be installed and assembled quickly and easily in an outdoor site such as a parking lot, playground, vacant lot, and the like in case of emergency.

Fourthly, if the module is thoroughly managed (disinfection, quarantine, etc.), there are no restrictions on the number of uses for installation/dismantlement, and in case of contamination or inoperability, only the relevant module can be replaced with new one, so no additional cost occurs.

Fifthly, it is possible to install multiple-stage structure, such as two-story, three-story, and the like, so that a large-scale room can be constructed in a limited space.

Sixthly, all the air supply/exhaust facilities and equipment for maintaining a negative pressure are operated by individual valves and controllers for each room, so even if an abnormality occurs in one room, the rest of rooms operates normally.

Seventhly, the above-described modular negative pressure isolation room is designed in a modular way to be loaded into a truck, enabling rapid transportation and movement.

Eighthly, as compared to negative pressure isolation facilities based on existing containers and tents, the above-described negative pressure isolation room has advantages of excellent air-tightness, free cooling/heating, and easy separation/discharge of sewage/wastewater without external contact.

The effects of the present disclosure are not limited to the above-mentioned effects, and other effects not mentioned above will be clearly understood by those skilled in the art from the description of the claims.

### BRIEF DESCRIPTION OF THE DRAWINGS

FIG. 1 is a plan view illustrating a field-type modular negative pressure isolation room system according to the first embodiment of the present disclosure;

FIG. 2 is a perspective view illustrating a part of the field-type modular negative pressure isolation room system according to the first embodiment of the present disclosure, and showing the inside of the room after a ceiling panel is removed;

FIG. 3 is a plan view illustrating the types of coatings formed on a base plate surface of a floor panel in the field-type modular negative pressure isolation room system according to the first embodiment of the present disclosure;

FIG. 4 is a combined perspective view illustrating a part of the field-type modular negative pressure isolation room system according to the first embodiment of the present disclosure;

FIGS. 5 to 8 are exploded perspective views illustrating a part of the field-type modular negative pressure isolation room system according to the first embodiment of the present disclosure;

FIG. 9 is a front view illustrating a part of the field-type modular negative pressure isolation room system according to the first embodiment of the present disclosure;

FIG. 10 is a plan view illustrating a part of the field-type modular negative pressure isolation room system according to the first embodiment of the present disclosure;

FIG. 11 is a perspective view illustrating a wall panel reinforcing-connecting means in the field-type modular negative pressure isolation room system according to the first embodiment of the present disclosure;

FIG. 12 is a perspective view illustrating a seal member formed on a connecting portion between inner wall panels, a connecting portion between outer wall panels, and a connecting portion between the inner wall panel and the outer wall panel in the field-type modular negative pressure isolation room system according to the first embodiment of the present disclosure;

FIGS. 13 and 14 are perspective views illustrating a bracket provided for connecting a ceiling and a wall panel and installed below a ceiling panel in a field-type modular negative pressure isolation room system according to the second embodiment of the present disclosure;

FIG. 15 is a bottom perspective view illustrating the bracket provided for connecting the ceiling and the wall panel in the field-type modular negative pressure isolation room system according to the second embodiment of the present disclosure;

FIG. 16 is a block diagram illustrating a method for constructing the field-type modular negative pressure isolation room system according to the first embodiment of the present disclosure; and

FIG. 17 is a block diagram illustrating a method for constructing the field-type modular negative pressure isolation room system according to the second embodiment of the present disclosure.

### BEST MODE FOR CARRYING OUT THE INVENTION

Hereinafter, preferred embodiments of the present disclosure will be described in detail with reference to the accompanying drawings so that those of ordinary skill in the art to which the present disclosure pertains can easily implement the present disclosure.

In the below description regarding the present embodiments, descriptions for technical contents which are well known in the technical field to which the present disclosure pertains and are not directly related to the present disclosure will be omitted. This is to more clearly convey the gist of the present disclosure without obscuring it by omitting unnecessary description.

For the same reason, some components in the accompanying drawings are exaggerated, omitted, or schematically illustrated.

In addition, a size of each component does not fully reflect an actual size thereof. In each figure, the same reference numerals are assigned to the same or corresponding elements.

In addition, it will be appreciated that expressions and predicates used herein with respect to terms (for examples, “front”, “back”, “up”, “down”, “top”, “bottom”, “left”, “right”, “lateral”) representing directions or the like of an apparatus or an element are used merely to simplify the description of the disclosure and do not indicate or imply that the related apparatus or element simply must have a particular direction.

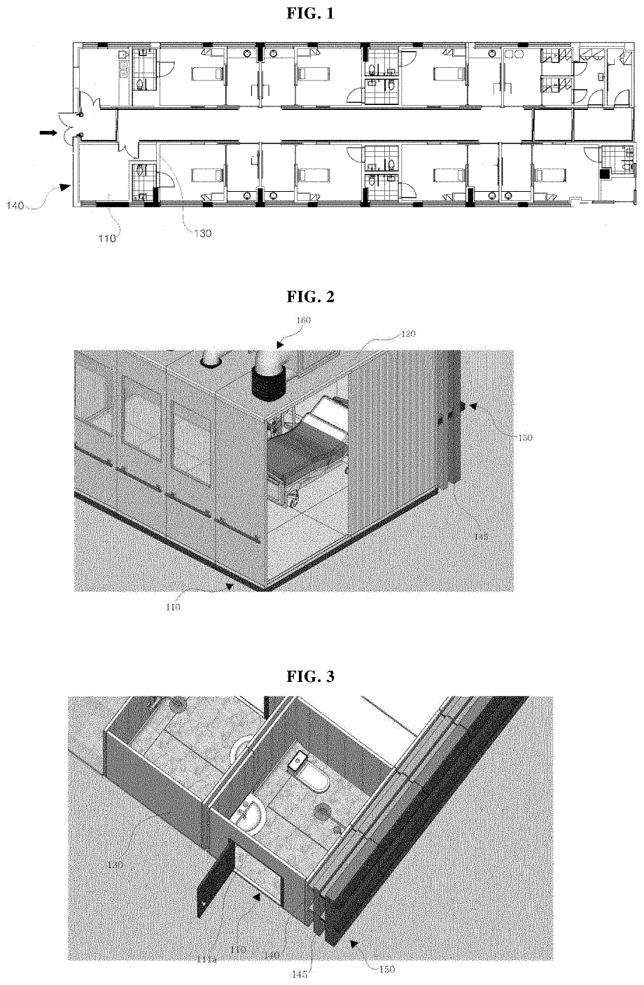

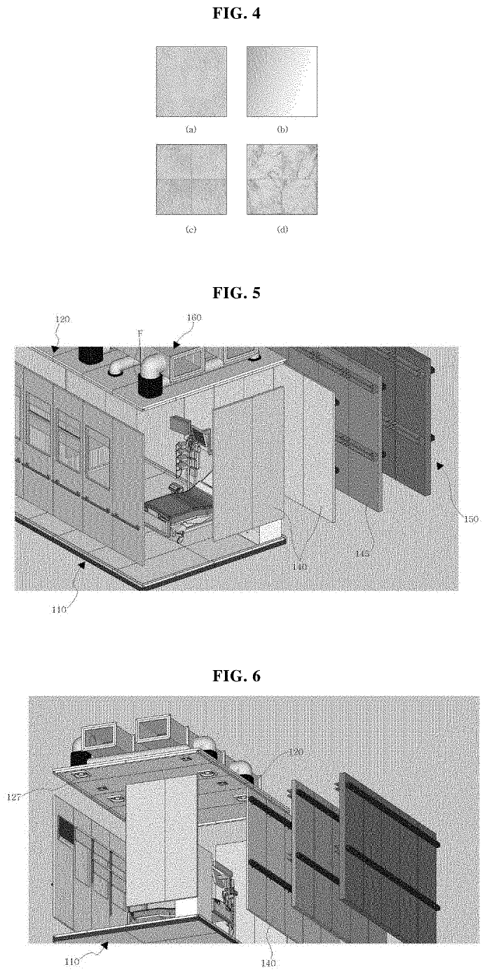

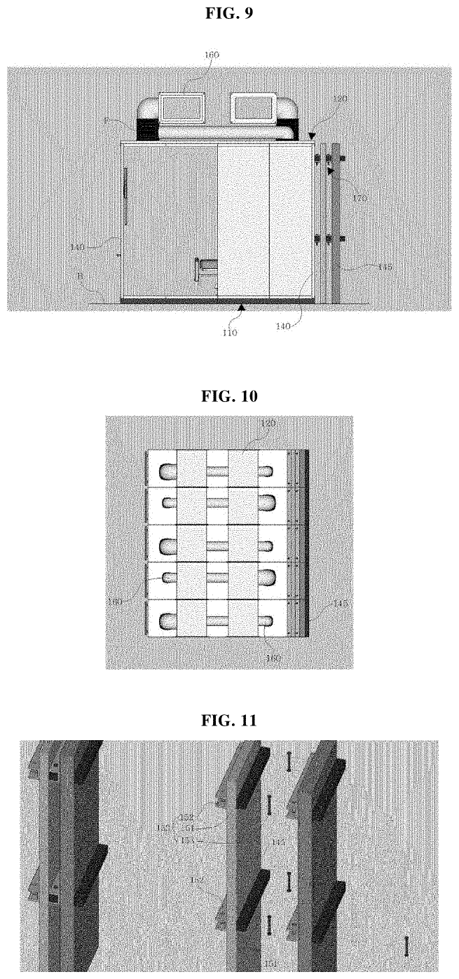

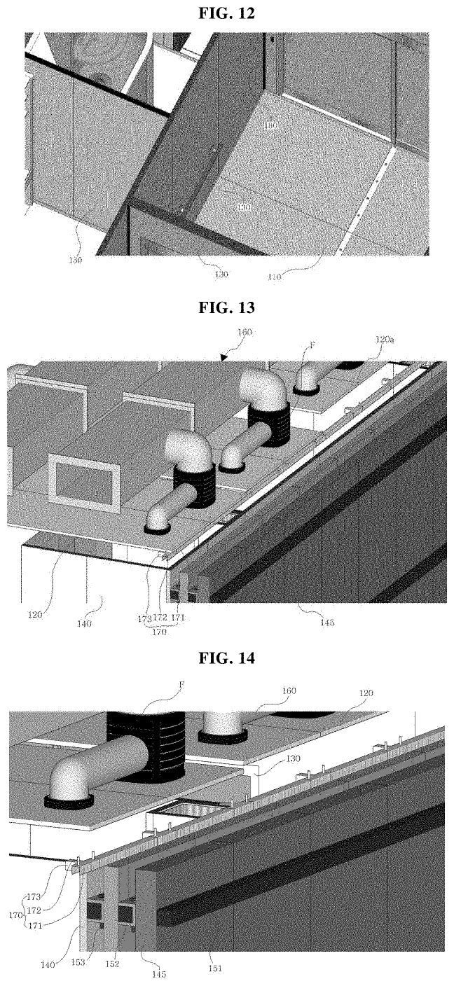

FIG. 1 is a plan view illustrating a field-type modular negative pressure isolation room system according to the first embodiment of the present disclosure, FIG. 2 is a perspective view illustrating a part of the field-type modular negative pressure isolation room system according to the first embodiment of the present disclosure, and showing the inside of the room after a ceiling panel is removed, FIG. 3 is a plan view illustrating the types of coatings formed on a base plate surface of a floor panel in the field-type modular negative pressure isolation room system according to the first embodiment of the present disclosure, FIG. 4 is a combined perspective view illustrating a part of the field-type modular negative pressure isolation room system according to the first embodiment of the present disclosure, FIGS. 5 to 8 are exploded perspective views illustrating a part of the field-type modular negative pressure isolation room system according to the first embodiment of the present disclosure, FIG. 9 is a front view illustrating a part of the field-type modular negative pressure isolation room system according to the first embodiment of the present disclosure, FIG. 10 is a plan view illustrating a part of the field-type modular negative pressure isolation room system according to the first embodiment of the present disclosure, FIG. 11 is a perspective view illustrating a wall panel reinforcing-connecting means in the field-type modular negative pressure isolation room system according to the first embodiment of the present disclosure, and FIG. 12 is a perspective view illustrating a seal member formed on a connecting portion between inner wall panels, a connecting portion between outer wall panels, and a connecting portion between the inner wall panel and the outer wall panel in the field-type modular negative pressure isolation room system according to the first embodiment of the present disclosure.

As illustrated in FIGS. 1 to 12, in the field-type modular negative pressure isolation room system according to the first embodiment of the present disclosure, an inner wall panel 130 and an outer wall panel 140 are installed between a floor panel 110 and a ceiling panel 120 to partition an internal space into a plurality of rooms, and the floor panel 110, the ceiling panel 120, the inner wall panel 130 and the outer wall panel 140 are assembled in a modular structure which may be dismantled.

Here, the modular structure refers to structures manufactured in a manufacturing plant for constructing various kinds of panels, a negative pressure isolation room satisfying the medical legal regulations, wherein the structures for negative pressure isolation room are employed in a way that they are usually stored in a warehouse, etc., and are assembled and installed using an outdoor site such as a parking lot, playground, vacant lot, and the like in case of emergency to utilize as a negative pressure isolation room, and they are disinfected and dismantled during demolition and stored in the warehouse again.

Referring to FIG. 1, the plurality of rooms may be formed as a front chamber, a negative pressure isolation room, a toilet, a mechanical equipment room, and the like, or may be formed as a vestibule for a hallway, a shower room and toilet for a medical staff, a sewage/waste disposal room, a utility and equipment storage room, a negative pressure hallway, an online visiting room, a negative pressure isolation room, a front chamber for a room, a toilet for a patient, a mechanical equipment room and the like.

The plurality of rooms illustrated in FIG. 1 is an example for explaining the field-type modular negative pressure isolation room system of the present disclosure, and is not limited thereto, and may be modified into various forms according to installation conditions or design conditions.

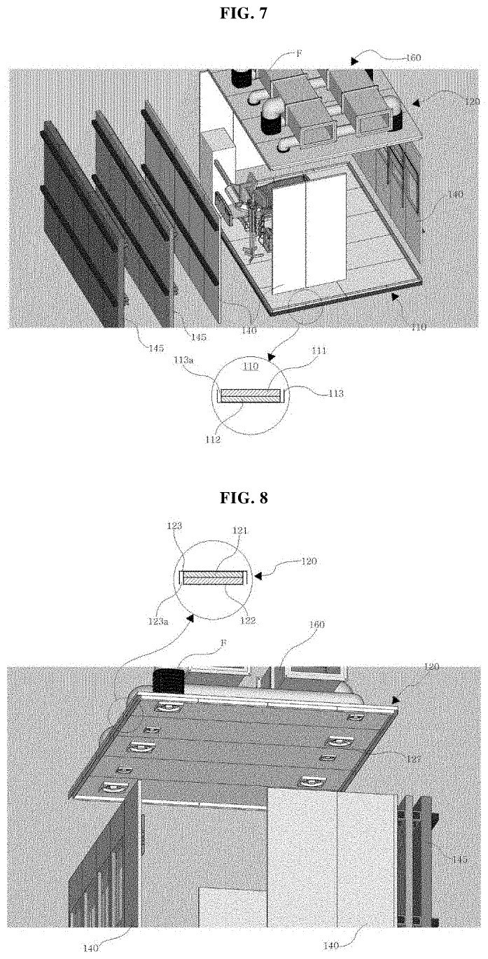

Referring to FIGS. 7 and 8, in the field-type modular negative pressure isolation room system according to the present disclosure, this system has a configuration in which all of the floor panel 110, the inner and outer wall panels 130, 140 and the ceiling panel 120 are inserted into and coupled to a “n” shaped or “U” shaped duct structure (groove structure), which is integrally formed with the ceiling structure 120, without separate fastening device, and a variety of medical equipment for active treatment and healing of patients, a door for securing airtightness, a measuring instrument, and the like may be integrally provided on the inner and outer wall panels 130 and 140. In addition, an air supply/exhaust facility 160 (including a high efficiency particulate air (HEPA) filter) and a negative pressure maintaining device (not shown) are integrally configured on the ceiling panel 120, and the above panels 110 to 140 are manufactured with the same standard to have a technical characteristic by which size and structure of a sickbed may be freely adjusted according to the situation.

First, referring to FIG. 7, in the field-type modular negative pressure isolation room system according to the first embodiment of the present disclosure, and the floor panel 110 includes a plurality of base plates 111, a plurality of floor plates 112, and a lower end fixing plate 113 having a fixing groove part 113a formed by bending it.

In more detail, in the floor panel 110, the plurality of base plates 111 are assembled adjacent to each other in a horizontal direction. A coating part 111a is formed on a surface of the base plate 111 (see FIGS. 3 and 4), and the coating part 111a is a metal type (a), a mirror type (b), a field block type (c), a marble type (d), and the like.

The floor plates 112 are installed on the base plate 111, and are assembled adjacent to each other in the horizontal direction.

The lower end fixing plate 113 is installed between the base plate 111 and the floor plate 112, and the fixing groove part 113a is formed at an edge portion by bending the lower end fixing plate so as to allow lower end portions of the inner wall panel 130 and the outer wall panel 140 to be inserted and coupled thereto.

The fixing groove part 113a formed by bending the lower end fixing plate is formed in a “U” shaped duct structure, and the lower end portions of the inner and outer wall panels 130 and 140 are inserted into and fastened to the fixing groove part.

And, referring to FIG. 8, the ceiling panel 120 includes a plurality of roof plates 121 positioned on an uppermost surface and assembled adjacent to each other in the horizontal direction; a plurality of ceiling plates 122 installed under the roof plate 121 and assembled adjacent to each other in the horizontal direction; and an upper end fixing plate 123 installed between the roof plate 121 and the ceiling plate 122, and having a fixing groove part 123a formed at an edge portion by bending the upper end fixing plate so as to allow upper end portions of the inner wall panel 130 and the outer wall panel 140 to be inserted and coupled thereto.

In more detail, in the ceiling panel 120, the plurality of roof plates 121 are located at an uppermost surface and assembled adjacent to each other in the horizontal direction. The ceiling plates 122 are installed under the roof plate 121, and are assembled adjacent to each other in the horizontal direction.

The upper end fixing plate 123 is installed between the roof plate 121 and the ceiling plate 122, and the fixing groove part 123a is formed at an edge portion by bending the upper end fixing plate so as to allow the upper end portions of the inner wall panel 130 and the outer wall panel 140 to be inserted and coupled thereto.

The fixing groove part 123a formed by bending the upper end fixing plate is formed in a “∩” shaped duct structure, and the upper end portions of the inner and outer wall panels 130 and 140 are inserted into and fastened to the fixing groove part.

In addition, referring to FIGS. 3, 5 and 11, a plurality of reinforcement wall panels 145 may be further installed on an outer surface of the outer wall panel 140 by a wall panel reinforcing-connecting means 150.

By further installing the reinforcement wall panel 145 to increase insulation and durability, it is possible to keep the interior warm in winter and cool in summer despite the field installation. Furthermore, the system of the present disclosure may be installed so that it is not affected by the surrounding climate even in very hot region or cold region.

When describing a configuration of the wall panel reinforcing-connecting means 150 in detail, the wall panel reinforcing-connecting means 150 includes a connection block 151 that is fixedly installed on any one of both outer surfaces of the reinforcement wall panel 145; a “ [symbol] ”-shaped connection bracket 152 fixedly installed on the other one of both outer surfaces of the reinforcement wall panel 145 so as to be coupled to the connection block 151; and a fastening bolt 153 passing through the “ [symbol] ”-shaped connection bracket 152 and the connection block 151 to fasten them to each other in a state in which the connection block 151 is inserted in a coupling groove 152a of the “ [symbol] ”-shaped connection bracket 152.

In addition, referring to FIGS. 8 to 10, the air supply/exhaust facilities 160 may be integrally installed on the ceiling panel 120, and the air supply/exhaust facilities 160 may be alternately arranged in a zigzag form on the ceiling panel 120, and a HEPA filter F and a backflow preventing damper (not shown) are installed on the air supply/exhaust facility 160.

The air supply/exhaust facility 160 in which the HEPA filter F is installed has a structure that is integrally installed on the ceiling panel 120. A blower fan 127 is installed at a position at which the air supply/exhaust facility 160 and the ceiling panel 120 are connected to each other (see FIG. 8).

In addition, referring to FIG. 12, seal member 180 may be installed at a connecting portion between the inner wall panels 130 and 130, a connecting portion between the outer wall panels 140 and 140, and a connecting portion between the inner wall panel 130 and the outer wall panel 140.

The seal member 180 may be made of a silicone material, and is in closely contact with the connecting portion between the inner wall panels 130 and 130, the connecting portion between the outer wall panels 140 and 140, and the connecting portion between the inner wall panel 130 and the outer wall panel 140 to serve to maintain airtightness.

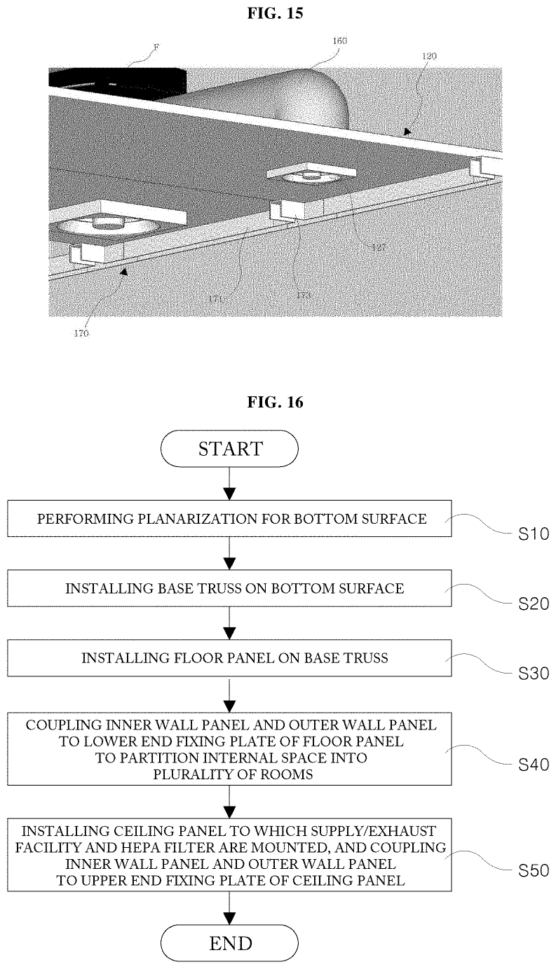

Meanwhile, FIGS. 13 and 14 are perspective views illustrating a bracket provided for connecting a ceiling and a wall panel and installed below a ceiling panel in a field-type modular negative pressure isolation room system according to the second embodiment of the present disclosure, and FIG. 15 is a bottom perspective view illustrating the bracket provided for connecting the ceiling and the wall panel in the field-type modular negative pressure isolation room system according to the second embodiment of the present disclosure.

Referring to FIGS. 13 to 15, in the field-type modular negative pressure isolation room system according to the second embodiment of the present disclosure, a bracket 170 for connecting the ceiling panel and the wall panel may be installed under the ceiling panel 120 so as to assemble the inner wall panel 130 and the outer wall panel 140 to the ceiling panel.

The bracket 170 for connecting the ceiling panel and the wall panel includes a first channel part 171 configured to allow the upper end portion of the outer wall panel 140 to be inserted thereto; a coupling protrusion 172 extending on an upper surface of the first channel part 171 to be coupled into a coupling hole 120a of the ceiling panel 120; and a second channel part 173 formed perpendicular to the first channel part 171 to allow the upper end portion of the inner wall panel 130 to be inserted thereinto. The bracket 170 for connecting the ceiling panel and the wall panel has a “T” shape.

That is, the upper end portion of the outer wall panel 140 is inserted into the first channel part 171, and the coupling protrusion 172 extends on the upper surface of the first channel part 171 to be coupled into the coupling hole 120a of the ceiling panel 120. The second channel part 173 is formed perpendicular to the first channel part 171 to allow the upper end portion of the inner wall panel 130 to be inserted thereinto.

Meanwhile, FIG. 16 is a block diagram illustrating a method for constructing the field-type modular negative pressure isolation room system according to the first embodiment of the present disclosure.

Referring to the above drawing, a method for constructing the field-type modular negative pressure isolation room system according to the first embodiment of the present disclosure includes the steps of performing planarization for a bottom surface (S10); installing a base truss B on the bottom surface (S20); installing the floor panel 110 on the base truss B (see FIG. 9) (S30); coupling the lower end portions of the inner wall panel 130 and the outer wall panel 140 into the fixing groove part 113a formed in the lower end fixing plate 113 of the floor panel 110 and installing vertically the inner wall panel 130 and the outer wall panel 140 to partition an internal space into the plurality of rooms (S40); and installing the ceiling panel 120 to which the air supply/exhaust facility 160 and the HEPA filter F are mounted, on the upper end portions of the inner wall panel 130 and the outer wall panel 140, and inserting and coupling the upper end portions of the inner wall panel 130 and the outer wall panel 140 into the fixing groove part 123a formed in the upper end fixing plate 123 of the ceiling panel 120 (S50).

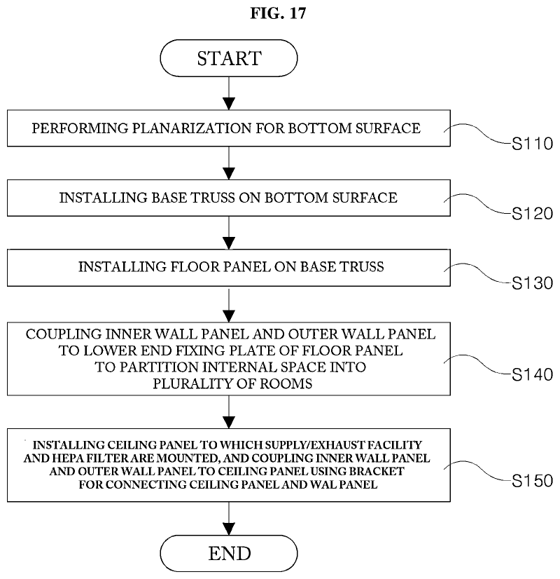

On the other hand, FIG. 17 is a block diagram illustrating a method for constructing the field-type modular negative pressure isolation room system according to the second embodiment of the present disclosure.

Referring to the above drawing, a method for constructing the field-type modular negative pressure isolation room system according to the second embodiment of the present disclosure includes the steps of performing planarization for the bottom surface (S110); installing the base truss B on the bottom surface (S120); installing the floor panel 110 on the base truss B (see FIG. 9) (S130); coupling the lower end portions of the inner wall panel 130 and the outer wall panel 140 into the fixing groove part 113a formed in the lower end fixing plate 113 of the floor panel 110 and installing vertically the inner wall panel 130 and the outer wall panel 140 to partition an internal space into the plurality of rooms (S140); and installing the ceiling panel 120 to which the air supply/exhaust facility 160 and the HEPA filter F are mounted, on the upper end portions of the inner wall panel 130 and the outer wall panel 140, and coupling the upper end portions of the inner wall panel 130 and the outer wall panel 140 to the ceiling panel using the bracket 170 for connecting the ceiling panel and the wall panel.

As described above, in this embodiment, the internal space is partitioned into the plurality of rooms by assembling the floor panel 110, the ceiling panel 120, the inner wall panel 130, and the outer wall panel 140, and wherein the floor panel 110, the ceiling panel 120, the inner wall panel 130, and the outer wall panel 140 are configured as the module, so assembly and disassembly (dismantlement) for the above panels may be repeatedly performed.

Also, by using the method in which the floor panel 100 is installed on the base truss B, the field-type modular negative pressure isolation room system according to the first embodiment of the present disclosure may be constructed not only in one-story structure, but also in multi-stage structure including two-story, three-story, and the like, so a large-scale room may be built in a limited space.

In addition, all the air supply/exhaust facilities and equipment for maintaining a negative pressure are operated by individual valves and controllers for each room, so even if an abnormality occurs in one room, the rest of rooms operates normally.

Furthermore, the modular negative pressure isolation room system is designed in a modular way to be loaded into a truck, enabling rapid transportation and movement.

Furthermore, as compared with conventional negative pressure isolation facility based on a container, a tent, or the like, the field-type modular negative pressure isolation room system according to the first embodiment of the present disclosure has advantages of excellent airtightness, free cooling/heating, and easy separation/discharge of sewage/wastewater without external contact.

For reference, the installation standards defined in the legal regulations applied to the field-type modular negative pressure isolation room system of the present disclosure are as follow: \textcircled{1} An area of 15 m² or more is secured, and this area should not include the areas for toilet and shower room. \textcircled{2} The front chamber should be installed, and a door for the negative pressure room should be installed. \textcircled{3} The doors of the negative pressure room and the front chamber should not be opened and closed at the same time. \textcircled{4} The toilet should be installed inside the negative pressure room. \textcircled{5} In an air supply facility, the HEPA filter should be installed at an air supply port for each room, but a backflow prevention damper should be provided. \textcircled{6} In an exhaust facility, the HEPA filter should be installed, and an exhaust damper for each room should be installed to prevent the spread of infection due to backflow, and air should be exhausted to the outside away from the air inlet and a zone where people gather. \textcircled{7} A differential pressure gauge and differential pressure indicator which are able to check the negative pressure at all times should be equipped, and an alarm sound should be generated in case of abnormality.

Also, the operating standards for the negative pressure isolation room defined in the legal regulations are as follow: \textcircled{1} The difference in negative pressure between a space with the negative pressure sickbed and the front chamber and between the negative pressure zone and non-negative pressure zone should be maintained at −2.5 Pa (−0.255 mmAq) or more, respectively. \textcircled{2} The number of ventilation for the room with the negative pressure sickbed and the front chamber entire room should be 6 times/hr or more. \textcircled{3} Sewage/drainage generated from the negative pressure zone should be discharged separately or after disinfection and sterilization.

In addition, the negative pressure difference in the negative pressure isolation room should be 25 Pa (−0.255 mmAq, 0.01 inch H20, 0.254926 mmH20, 0.018752 mmHg) or more, so the negative pressure isolation room is required to have ACH (air change per hour) ratio, which means the number of exchanges of fresh air per hour, of 12 or more, filter performance capable of recovering 99.97% or more of particles of 0.3 μm or larger, and airflow in the direction towards the patient in the room.

In addition, for the air-conditioning facility of the negative pressure isolation room defined in the legal regulations, \textcircled{1} An exclusive supply/exhaust facility should be established to distinguish it from an exhaust facility provided for another purpose. \textcircled{2} A system should be provided that does not cause spread of infection or cross-contamination due to backflow of air even when the air conditioning system is stopped due to a power outage or mechanical breakdown. \textcircled{3} An appropriate temperature and humidity maintenance system should be installed so that the patient may live in the hospital without opening a window, but a fan coil, a system air conditioner, and the like, where bacterial can grow in in summer, should not be installed. \textcircled{4} The room should be designed such that the noise therein is 50 dB(A) or less. \textcircled{5} The air conditioning facility such as air conditioners, an exhaust fan (filter), and the like should be designed to be linked with a standby facility. \textcircled{6} The air conditioning facility supplied to the negative pressure sickbed should be designed in a structure that does not cause power failure.

Furthermore, in the method for supplying air in the negative pressure isolation room, \textcircled{1} A dedicated air supply/exhaust system must be provided, and supplying air should be performed in an all-outdoor (heat recovery air conditioner) manner. \textcircled{2} Air discharged from the room and the front chamber should be filtered with a HEPA and then discharged the outside such that discharged air is not recirculated in other sickbeds. \textcircled{3} The number of ventilation should be 6 to 12 times/hr. \textcircled{4} The HEPA filter and an airtight back draft damper should be installed at the air supply/exhaust port of the room to prevent the backflow of contaminated air against stoppage of air conditioning and the like. \textcircled{5} The room should be designed so that the air supply system and the exhaust system are interconnected, so that when the exhaust system is halted, the room should not have positive pressure compared to the adjacent “rooms” due to a sudden pressure change. \textcircled{6} The air supply system should be designed to be automatically turned off when the exhaust system is malfunctioned or halted and to be re-operated when the standby exhaust fan is operated and the pressure is stabilized.

In addition, in the exhaust method for the negative pressure isolation room, \textcircled{1} The exhaust port should discharge entire amount of air to the outside through a filter having HEPA filter grade or higher. \textcircled{2} The exhaust port should be as close to the patient's respiratory tube as possible. \textcircled{3} The exhaust port should be installed at a location near the patient's head or at a lower end portion of a wall. \textcircled{4} An exhaust duct extending each room should exhaust air independently, and an exhaust fan should be installed at an end of the exhaust duct. \textcircled{5} For the exhaust fan in the negative pressure isolation zone, a spare fan should be provided to prepare for breakdown of the exhaust fan. \textcircled{6} The exhaust port extending outside a building should be located at a point away at least 2 m from the ground to prevent air from being directly exhausted to nearby people, and there should be no inlet for another system within 2 m. It is recommended to install the exhaust port such that a direction of the exhaust port does not face the inlet of another system. \textcircled{7} The facility having sufficient capacity than that necessary to maintain the predetermined negative pressure should be installed. \textcircled{8} The exhaust fan in the negative pressure isolation zone should be connected to an uninterruptible power supply (UPS) and an emergency generator so that exhaust can be performed even when a power outage occurs.

Furthermore, in the method for controlling the negative pressure in the negative pressure isolation room, \textcircled{1} Indoor air pressure should be adjusted so that the air flows from an area with a low pollution level to an area with a high pollution level. For example, in Example 1), air flows in the order of non-negative pressure isolation zone>a vestibule for a hallway (dressing)>negative pressure hallway>front chamber for room>room>toilet. In Example 2), After showing, air flow in the order of wearing room>shower room>protective cloth-dressing room>decontamination room>equipment storage room>internal hallway. \textcircled{2} In order to stably control indoor air pressure in the negative pressure room, etc., the air supply port and the exhaust port for the negative pressure sickbed and room-the front chamber should be provided, but in a case of a toilet in a separate room, only the exhaust port is installed. \textcircled{3} Differential pressure indicators indicating the differences in pressure of rooms, such as a toilet, a room, a front chamber of room, an internal hallway, and the like in units of −2 Pa, respectively, should be provided. \textcircled{4} A differential pressure indicator displaying a differential pressure to one decimal place (0.1 Pa) is installed at an exit of the room in which the negative pressure is maintained (However, if the differential pressure of 4 Pa or more between rooms in the negative pressure zone is secured, a differential pressure indicator in units of 1 Pa is installed). \textcircled{5} An air conditioning controller should be installed in a central control room, etc., so that the negative pressure in the negative pressure isolation zone is properly maintained. \textcircled{6} No one other than a manager should be able to manipulate the air conditioning controller. In the event of an abnormality, the manager and medical staff must take immediate action through an alarm sound, and when a malfunction occurs, it should be recorded automatically.

In addition, in an installation of sanitary fixture, \textcircled{1} A hand washing appliance should be usable for washing a face, etc., and should have a configuration to prevent water from splashing and a size by which a wrist is sufficiently submerged in water. \textcircled{2} A faucet in the sanitary fixture should be installed in a structure that can be used without touching it (a non-contact type automatic faucet). \textcircled{3} A connection between a water supply pipe and a toilet bowl should be made so that no backflow occurs into the water supply pipe. \textcircled{4} A container for detergent and disinfectant should be installed above the hand washing appliance to avoid contamination on a top plate of a counter washbasin, a wall, and the like when detergent or disinfectant is used.

Also, for the drainage equipment, \textcircled{1} Drain pipe and vent pipe connected to a hand washing container or a toilet bowl should be installed such that drainage does not flow backwards. \textcircled{2} A drain pipe in the negative pressure isolation zone should be installed separately up to a dedicated wastewater tank and then merged with another wastewater treatment facility after performing disinfection or sterilization. \textcircled{3} A wastewater treatment system should be installed suitable for chemical or heat treatment. \textcircled{4} A vent pipe should be installed in a wastewater tank to prevent backflow of wastewater, and a sterilizing filter is installed at one end of the vent pipe. \textcircled{5} A facility and verification ports for biological inactivation of microorganisms should be installed. \textcircled{6} An overflow preventing barrier for preventing overflow in an emergency should be installed in a dedicated high-temperature/high-pressure sterilizing wastewater tank within a negative pressure isolation zone.

In addition, for a firefighting facility in the negative pressure isolation room, \textcircled{1} Fire hydrants, fire extinguishers (clean fire extinguishing agents) and the like should be provided (outside the negative pressure room) against fire. \textcircled{2} A simple sprinkler facility should be installed against fire. \textcircled{3} In the event of a fire, all interlocks in the negative pressure isolation sickbed and the isolation zone should be configured to be automatically and manually released and opened. \textcircled{4} A fire extinguisher should be provided in the front of each room. A firefighting equipment shall be a simple fire extinguishing device.

In addition, for a medical gas equipment of the negative pressure isolation room, \textcircled{1} Oxygen and compressed air should be supplied from the outside (a simple utility room) through piping. \textcircled{2} An outlet box for the medical gas outlet box should be configured to be installed on a wall in a state exposed. \textcircled{3} A medical gas equipment should be configured such that other patients are not infected through a suction equipment (installation of HEPA Filter). \textcircled{4} On the basis of seven (7) sickbeds, the utility room should be equipped with two (reserve) vacuum pumps, an oxygen supply device, and the like. \textcircled{5} A spare medical gas supply equipment and a spare vacuum pump should be provided so that t it is possible to perform functions thereof even when a medical gas supply equipment and a vacuum pump have a breakdown. \textcircled{6} The utility room should be equipped with a device such that no one other than authorized persons can enter.

Furthermore, in a method for supplying electricity using an electrical installation, electricity is supplied by Korean Electric Power Corporation. A simple sprinkler facility should be installed against fire. Uninterruptible power supply (UPS) should be supplied in case of power outage. In case of power outage, power is supplied to an emergency power generation system connected in parallel with the UPS. At least two generators are constructed to secure a backup power supply source.

In addition, in the construction of the electrical installation, \textcircled{1} A partially color-coded wiring should be used in order to enable load balance to be checked. \textcircled{2} A separation distance between communication lines should be secured to prevent various measuring instrument errors. \textcircled{3} Voltage below 300V: separation distance of 6 cm (12 cm or more at a location that is not well visible). \textcircled{3} Voltage greater than 300V or more: separation distance of 15 cm or more (30 cm or more at a location that is not well visible). \textcircled{5} If strong current wires are employed as cable, they should be installed not to be in contact with each to each other. \textcircled{6} A connection between external power lines is treated with a rain-proof type concentric plug having anti-fouling/water-proofing properties.

As described above, the present disclosure has the following advantages.

Firstly, the above-described negative pressure isolation room is a negative pressure isolation room which can be quickly installed for preparing the spread of infectious diseases and disassembled, as compared to a conventional negative pressure isolation room installed in a hospital, a construction cost therefor is low, and concerns about secondary infection in a hospital is low.

Secondly, the above-described negative pressure isolation room is a “mobile modular negative pressure room” which can be installed once a certain space is secured, and can be installed and dismantled in a region with low population density, so it is possible to minimize the occurrence of nimby phenomena, such as resistance of the residents caused by designating the existing facility as a “designated facility (hospital) for COVID 19”.

Thirdly, all main facilities necessary for constructing the negative pressure isolation room defined by the legal regulations are manufactured in a plant in a modular way, so the production process for the facilities is fast, and these facilities are normally dismantled and stored in warehouses, etc., and can be installed and assembled quickly and easily in an outdoor site such as a parking lot, playground, vacant lot, and the like in case of emergency.

Fourthly, if the module is thoroughly managed (disinfection, quarantine, etc.), there are no restrictions on the number of uses for installation/dismantlement, and in case of contamination or inoperability, only the relevant module can be replaced with new one, so no additional cost occurs.

Fifthly, it is possible to install multiple-stage structure, such as two-story, three-story, and the like, so that a large-scale room can be constructed in a limited space.

Sixthly, all the air supply/exhaust facilities and equipment for maintaining a negative pressure are operated by individual valves and controllers for each room, so even if an abnormality occurs in one room, the rest of rooms operates normally.

Seventhly, the above-described modular negative pressure isolation room is designed in a modular way to be loaded into a truck, enabling rapid transportation and movement.

Eighthly, as compared to negative pressure isolation facilities based on existing containers and tents, the above-described negative pressure isolation room has advantages of excellent air-tightness, free cooling/heating, and easy separation/discharge of sewage/wastewater without external contact.

On the other hand, in the present specification and drawings, preferred embodiments of the present disclosure have been disclosed, and although specific terms are used, these are only used in a general sense to easily explain the technical content of the present disclosure and to help the understanding of the present disclosure, and it is not intended to limit the scope of the disclosure. In addition to the embodiments disclosed herein, it is apparent to those of ordinary skill in the art that other modified examples based on the technical spirit of the present disclosure may be implemented.

The present disclosure may be variously modified and may take various forms. However, it should be understood that the present disclosure is not limited to the particular form recited in the foregoing detailed description, but rather that the present disclosure encompasses all modifications and equivalents and substitutes falling within the spirit and scope of the disclosure as defined by the appended claims.

1. A field-type modular negative pressure isolation room system, characterized in that an inner wall panel and an outer wall panel are installed between a floor panel and a ceiling panel to partition an internal space into a plurality of rooms, and the floor panel, the ceiling panel, the inner wall panel and the outer wall panel are assembled in a modular structure which may be dismantled.

2. The field-type modular negative pressure isolation room system of claim 1, wherein the floor panel comprising:
a plurality of base plates assembled adjacent to each other in a horizontal direction;
a plurality of floor plates installed on the base plate and assembled adjacent to each other in the horizontal direction; and
a lower end fixing plate installed between the base plate and the floor plate, and having a fixing groove part formed at an edge portion by bending the lower end fixing plate so as to allow lower end portions of the inner wall panel and the outer wall panel to be inserted and coupled thereto.

3. The field-type modular negative pressure isolation room system of claim 1, wherein the ceiling panel comprising:
a plurality of roof plates positioned on an uppermost surface and assembled adjacent to each other in the horizontal direction;
a plurality of ceiling plates installed under the roof plate and assembled adjacent to each other in the horizontal direction; and
an upper end fixing plate installed between the roof plate and the ceiling plate, and having a fixing groove part formed at an edge portion by bending the upper end fixing plate so as to allow upper end portions of the inner wall panel and the outer wall panel to be inserted and coupled thereto.

4. The field-type modular negative pressure isolation room system of claim 1, characterized in that a plurality of reinforcement wall panels are installed on an outer surface of the outer wall panel by a wall panel reinforcing-connecting means.

5. The field-type modular negative pressure isolation room system of claim 4, wherein the wall panel reinforcing-connecting means comprising:
a connection block that is fixedly installed on any one of both outer surfaces of the reinforcement wall panel;
a “ [symbol] ”-shaped connection bracket fixedly installed on the other one of both outer surfaces of the reinforcement wall panel so as to be coupled to the connection block; and
a fastening bolt passing through the “ [symbol] ”-shaped connection bracket and the connection block to fasten them to each other in a state in which the connection block is inserted in a coupling groove of the “ [symbol] ”-shaped connection bracket.

6. The field-type modular negative pressure isolation room system of claim 1, characterized in that air supply/exhaust facilities are alternately arranged in a zigzag form on the ceiling panel.

7. The field-type modular negative pressure isolation room system of claim 1, further comprising a bracket for connecting the ceiling panel and the wall panel may be installed under the ceiling panel so as to assemble the inner wall panel and the outer wall panel to the ceiling panel.

8. The field-type modular negative pressure isolation room system of claim 7, wherein the bracket for connecting the ceiling panel and the wall panel comprising:
a first channel part configured to allow an upper end portion of the outer wall panel to be inserted thereto;
a coupling protrusion extending on an upper surface of the first channel part to be coupled into a coupling hole of the ceiling panel; and
a second channel part formed perpendicular to the first channel part to allow an upper end portion of the inner wall panel to be inserted thereinto.

9. The field-type modular negative pressure isolation room system of claim 8, characterized in that the bracket for connecting the ceiling panel and the wall panel has a T shape.

10. The field-type modular negative pressure isolation room system of claim 1, characterized in that a seal member is installed at a connecting portion between the inner wall panels, a connecting portion between the outer wall panels, and a connecting portion between the inner wall panel and the outer wall panel.

11. The field-type modular negative pressure isolation room system of claim 1, wherein the plurality of rooms comprises a front chamber, a negative pressure isolation room, a toilet, and a mechanical equipment room.

12. The field-type modular negative pressure isolation room system of claim 2, wherein the rooms comprise a vestibule for a hallway, a shower room and toilet for a medical staff, a sewage/waste disposal room, a utility and equipment storage room, a negative pressure hallway, an online visiting room, a negative pressure isolation room, a front chamber for a room, a toilet for a patient, a mechanical equipment room.

13. A method for constructing a field-type modular negative pressure isolation room, comprising:
performing planarization for a bottom surface;
installing a base truss on the bottom surface;
installing a floor panel on the base truss;
coupling lower end portions of an inner wall panel and an outer wall panel into a fixing groove part formed in a lower end fixing plate of the floor panel and installing vertically the inner wall panel and the outer wall panel to partition an internal space into the plurality of rooms; and
installing a ceiling panel to which an air supply/exhaust facility and a HEPA filter are mounted, on the upper end portions of the inner wall panel and the outer wall panel, and inserting and coupling the upper end portions of the inner wall panel and the outer wall panel into the fixing groove part formed in the upper end fixing plate of the ceiling panel.

14. A method for constructing a field-type modular negative pressure isolation room, comprising:
performing planarization for the bottom surface;
installing a base truss on the bottom surface;
installing a floor panel on the base truss;
coupling lower end portions of an inner wall panel and an outer wall panel into a fixing groove part formed in a lower end fixing plate of the floor panel and installing vertically the inner wall panel and the outer wall panel to partition an internal space into the plurality of rooms; and
installing a ceiling panel to which an air supply/exhaust facility and a HEPA filter are mounted, on the upper end portions of the inner wall panel and the outer wall panel, and coupling the upper end portions of the inner wall panel and the outer wall panel to the ceiling panel using the bracket for connecting the ceiling panel and the wall panel.

15. The method for constructing field-type modular negative pressure isolation room of claim 13, characterized in that the inner space is partitioned into the plurality of rooms by assembling the floor panel, the ceiling panel, the inner wall panel, and the outer wall panel, wherein the floor panel, the ceiling panel, the inner wall panel, and the outer wall panel are configured as the module, so assembly and disassembly for the above panels may be repeatedly performed.

16. The method for constructing field-type modular negative pressure isolation room of claim 14, characterized in that the inner space is partitioned into the plurality of rooms by assembling the floor panel, the ceiling panel, the inner wall panel, and the outer wall panel, wherein the floor panel, the ceiling panel, the inner wall panel, and the outer wall panel are configured as the module, so assembly and disassembly for the above panels may be repeatedly performed.

17. The field-type modular negative pressure isolation room system of claim 5, characterized in that air supply/exhaust facilities are alternately arranged in a zigzag form on the ceiling panel.
